(19) United States
(12) Patent Application Publication
(10) Pub. No.: US 2007/0032451 A1
(43) Pub. Date: Feb. 8, 2007

(54) Blood soluble drag reducing hyaluronic acid

(76) Inventors: Kipling Thacker, Excelsior, MN (US); Marina Kameneva, Pittsburgh, PA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/364,566

(22) Filed: Feb. 27, 2006

Related U.S. Application Data
(60) Provisional application No. 60/657,119, filed on Feb. 28, 2005.

(51) Int. Cl. A61K 31/728

(52) U.S. Cl. 514/54

(57) ABSTRACT

The use of hyaluronic acid and physiologically acceptable salts thereof as drag reducing agents is described. The compositions of the invention can be used to increase aortic blood flow, increase arterial blood flow, increase venous blood flow, decrease blood pressure, decrease peripheral vascular resistance, diminish the development of atherosclerosis, and/or prevent lethality of hemorrhagic shock.

### CROSS REFERENCE TO RELATED APPLICATIONS

This application claims benefit under 35 U.S.C. §119(e) to application U.S. Ser. No. 60/657,119, filed Feb. 28, 2005 (Attorney docket number 186327/US), entitled “Blood Soluble Drag Reducing Hyaluronic Acid”, the contents of which are incorporated herein by reference in their entirety for all purposes.

### FIELD OF THE INVENTION

The present invention relates to improved microflow drag reducing factors for use in blood as well as the restoration and/or enhancement of microcirculation and tissue oxygenation. The invention is further directed to methods for the restoration and/or enhancement of microcirculation and perfusion and oxygenation of mammalian tissues through contacting such tissues with microflow drag reducing factors provided herein.

### BACKGROUND OF THE INVENTION

Drag reducing polymers (DRPs) provide positive hemodynamic effects in various acute and chronic animal models. Nanomolar concentrations of various DRPs that are injected intravenously have been shown to increase aortic and arterial blood flow and decrease blood pressure and peripheral vascular resistance. Intravenous injections of DRPs have also been shown to diminish the development of atherosclerosis in atherogenic animal models.

The DRPs that have been studied thus far have been polyacrylamides, polyethylene oxides, polyethylene glycols, a polysaccharide extracted from okra and calf thymus DNA. None of the DRPs studied have been a material that is found as a naturally occurring endogenous material of the living mammal.

Therefore, a need exists for the identification of an endogenous material of a mammal that can be used as a DRP, at an increased concentration greater than that found in the natural state of the mammal, such that the DRP can provide one of more beneficial effects to the mammal.

### BRIEF SUMMARY OF THE INVENTION

The present invention provides an endogenously derived DRP that can be used to increase aortic blood flow, arterial blood flow, increase venous blood blow, decrease blood pressure, decrease peripheral vascular resistance, diminish the development of atherosclerosis, and/or prevent lethality of hemorrhagic shock. Suitable DRPs of the present invention include hyaluronic acid and hyaluronic acid derivatives, such as pharmaceutically acceptable salts of hyaluronic acid.

The present invention further provides suitable pharmaceutical compositions of the DRPs of the invention.

Additionally, the present invention also provide packaged pharmaceutical formulations that contain the DRPs of the invention and instructions how to use the DRP(s).

The DRPs of the invention generally have molecular weights from those of oligosaccharides to about 7,000,000 kD, more particularly between about 500 kD and about 2000 kD. Useful concentrations of the DRPs are between about 10 ppm and about 1000 ppm.

### BRIEF DESCRIPTION OF THE DRAWINGS

FIG. 1 demonstrates that suitable concentrations of low molecular sodium hyaluronate are effective in reducing the flow resistance in an aqueous solution simulating behavior in blood.

### DETAILED DESCRIPTION

The present invention provides a unique and unexpected advantage that increased concentrations of an endogenously occurring material, relative to the naturally occurring level of such an endogenous material, can be used as a drag reducing polymer (DRP). Surprisingly, such an endogenous material is hyaluronic acid (or physiologically acceptable salts thereof), hereinafter referred to as “HA”. HA provides the unique advantage that the physiology of the mammal that requires treatment can more readily accept an endogenous substance rather than a foreign material, such as a polyethylene oxide.

The term hyaluronic acid is known in the art and it should be understood, that the term “hyaluronic acid” includes hyaluronan. Hyaluronic acid, under physiological conditions, is converted into various forms, based on electrolytes and other physiological medium. Therefore, it should be understood that once the hyaluronic acid is placed in an electrolytic solution, it is more correctly known as hyaluronan.

HA is a carboxyl containing polysaccharide. Carboxyl containing polysaccharides useful to treat the various diseases or conditions identified throughout the application are considered within the scope fo the present invention.

The term “a carboxyl-containing polysaccharide” is intended to mean a polysaccharide containing at least one carboxyl group. The polysaccharide chosen may initially contain carboxyl groups or it may be derivatized to contain carboxyl groups. Examples of carboxyl-containing polysaccharides include, but are not limited to, carboxymethyl cellulose, carboxymethyl chitin, carboxymethyl chitosan, carboxymethyl starch, alginic acid, pectin, carboxymethyl dextran, and glucosaminoglycans such as heparin, heparin sulfate, chondroitin sulfate and hyaluronic acid (HA). The most preferred carboxyl-containing polysaccharides are carboxymethyl cellulose, carboxymethyl chitin and HA. The most preferred carboxyl-containing polysaccharide is HA.

The compositions of the invention include a carboxyl-containing polysaccharide, or alternatively, a pharmacologically acceptable salt of the polysaccharide can be used, e.g., hyaluronan. Suitable pharmacologically acceptable salts are alkali or alkaline earth metal salts. Therefore, in one embodiment, the composition contains sodium hyaluronate.

Carboxyl-containing polysaccharides that can be used to prepare useful compositions of the invention are known compounds that are described, for example, in U.S. Pat. No. 4,517,295 and U.S. Pat. No. 4,141,973; and Handbook of Water Soluble Gums and Resins, Chapter 4, by Stelzer & Klug, published by McGraw-Hill, 1980. Processes for preparing the carboxyl-containing polysaccharide, HA, are illustrated in the Balazs patent, which details a procedure for extracting HA from rooster combs, and in U.S. Pat. No. 4,517,295 that describes fermentation process for making HA. The HA used to make the DRP should be highly purified (medical grade quality) for invasive applications.

The phrase “physiologically acceptable salts thereof (of hyaluronic acid)” is intended to include those derivatives wherein one or more of the acidic protons of the carboxylic acid groups of the hyaluronic acid moiety is substituted by a counterion. Suitable counterions include groups I, II, III and IV metals, ammonium complexes, amino acid complexes, etc. For example, the physiologically acceptable salt can include sodium, lithium, magnesium, potassium, ammonium ion and various amino acids as counterions.

The HA generally has a molecular weight of from oligosaccharides of HA to about 7,000,000 kD, in particular between about 500 kD and about 200 kD, more particularly between about 600 kD and 1700 kD and in one embodiment about 1100 kD. In one embodiment, the HA was a sodium salt.

HA can be provided in the form of a pharmaceutical composition. In one particular aspect, the pharmaceutical composition can be in the form of an injectable intravenous preparation. In another aspect, the composition can be placed into an intravenous solution that is administered over a period of time, e.g., an iv drip.

The pharmaceutical composition can be an aqueous solution that includes sodium salt(s), i.e., sodium chloride, potassium salt(s), i.e., potassium chloride, calcium salt(s), i.e., calcium chloride, magnesium salt(s), such as magnesium chloride, sodium acetate, sodium citrate and/or sodium phosphate. Alternatively, the solution can be a saline solution or PBS.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the HA may be dried by any art-known technique, such as lyophilization, and reconstituted prior to use.

For prolonged delivery, the HA can be formulated as a depot preparation for administration by implantation or intramuscular injection. The HA may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the HA for percutaneous absorption may be used. To this end, permeation enhancers may be used to facilitate transdermal penetration of the active compound(s). Suitable transdermal patches are described in for example, U.S. Patent No. 5,407,713.; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

The pharmaceutical compositions may, if desired, be presented in a pack or dispenser device which may contain one or more unit dosage forms containing the HA. The pack may, for example, comprise metal or plastic foil, such as a blister pack. The pack or dispenser device may be accompanied by instructions for administration.

The HA and pharmaceutical compositions described herein can be administered to achieve the intended result, for example in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of HA to a patient suffering from trauma ameliorates the effect of the loss of blood, possibly by improved peripheral oxygenation. Therapeutic benefit also includes halting or slowing the progression of the disease, regardless of whether improvement is realized.

For prophylactic administration, the HA may be administered to a patient at risk of developing one of the previously described diseases. Alternatively, prophylactic administration may be applied to avoid the onset of symptoms in a patient diagnosed with the underlying disorder.

The amount of HA administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Effective dosages may be estimated initially from in vitro assays. For example, an initial dosage for use in animals may be formulated to achieve a circulating blood or serum concentration of HA that is at or above an IC₅₀ of the HA as measured in as in vitro assay, such as those described in Kamevena, cited hrerein below, and those references cited therein. Calculating dosages to achieve such circulating blood or serum concentrations taking into account the bioavailability of the HA is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pagamonon Press, and the references cited therein.

Initial dosages can also be estimated from in vivo data, such as animal models. Animal models useful for testing the efficacy of compounds to treat or prevent the various diseases described above are well-known in the art.

Dosage amounts of the HA will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma levels of the HA which are sufficient to maintain therapeutic or prophylactic effect. For example, the HA may be administered once per week, several times per week (e.g., every other day), once per day or multiple times per day, depending upon, among other things, the mode of administration, the specific indication being treated and the judgment of the prescribing physician.

Preferably, the HA will provide therapeutic or prophylactic benefit without causing substantial toxicity. Toxicity of the HA may be determined using standard pharmaceutical procedures. The dose ratio between toxic and therapeutic (or prophylactic) effect is the therapeutic index.

The DRPs of the present invention can be used to increase aortic blood flow, increase arterial blood flow, increase venous blood flow, decrease blood pressure, decrease peripheral vascular resistance, diminish the development of atherosclerosis, and/or prevent lethality of hemorrhagic shock. The DRPs are provided in therapeutically effective amounts.

The DRPs of the invention can be evaluated using standard methods in the art to determine the efficacy in treatment of increase aortic blood flow, increase arterial blood flow, increase venous blow flow, decrease blood pressure, decrease peripheral vascular resistance, diminish the development of atherosclerosis, and/or prevent lethality of hemorrhagic shock. Suitable animal models are known as described by Kameneva et al. “Blood soluble drag-reducing polymers prevent lethality from hemorrhagic shock in acute animal experiments”, Biorheology 41 (2004), 53-64, the contents of which are incorporated herein in their entirety, including the teachings of those references cited therein.

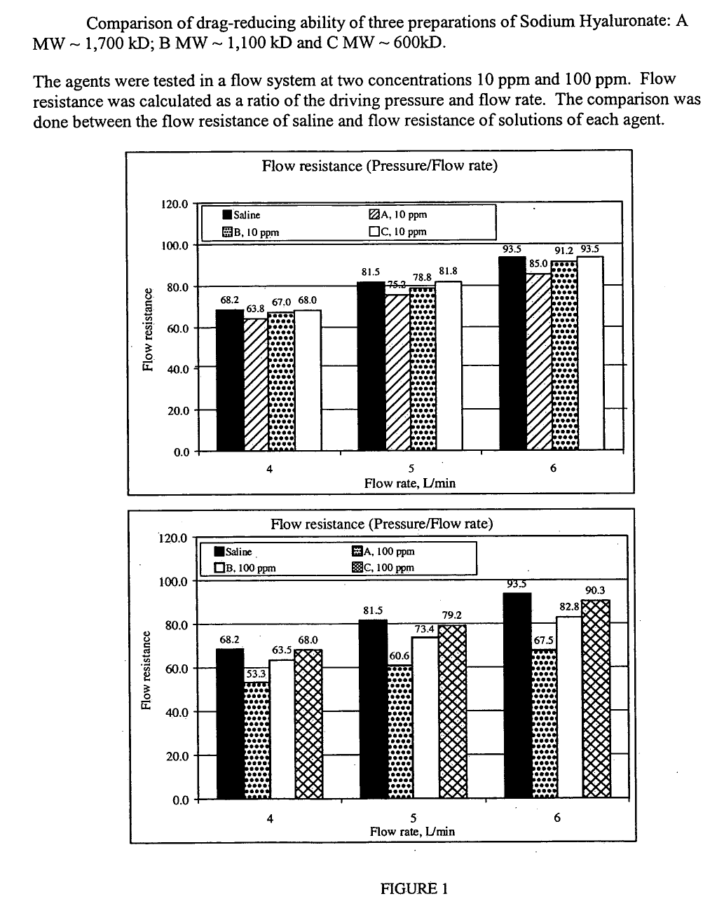

FIG. 1 provides graphical evidence that sodium hyaluronate is useful to reduce the viscosity of an aqueous solution that is a suitable model predictive of efficacy in blood.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

I claim:

1. A blood soluble drag reducing composition, comprising hyaluronic acid or a physiologically acceptable salt thereof and a pharmaceutically acceptable carrier.

2. The blood soluble drag reducing composition of claim 1, wherein the physiologically acceptable salt of hyaluronic acid is a sodium salt.

3. The blood soluble drag reducing composition of claim 1, wherein the molecular weight of the hyaluronic acid or physiologically acceptable salt is from that of an oligosaccharide to about 7,000,000 kD.

4. The blood soluble drag reducing composition of claim 3, wherein the physiologically acceptable salt of hyaluronic acid is a sodium salt.

5. The blood soluble drag reducing composition of claim 4, wherein the molecular weight of the sodium salt of hyaluronic acid is between about 600 kD and about 1700 kD.

6. The blood soluble drag reducing composition of claim 5, wherein the concentration of the sodium salt of hyaluronic acid is between about 10 ppm and about 1000 ppm in the blood.

7. A method to increase aortic blood flow, increase arterial blood flow, increase venous blood flow, decrease blood pressure, decrease peripheral vascular resistance, diminish the development of atherosclerosis, and/or prevent lethality of hemorrhagic shock, comprising the step of administering a therapeutically acceptable amount of hyaluronic acid or a physiologically acceptable salt thereof to a subject in need thereof.

8. The method of claim 7, wherein the physiologically acceptable salt of hyaluronic acid is a sodium salt.

9. The method of claim 7, wherein the molecular weight of the hyaluronic acid or physiologically acceptable salt thereof is from that of an oligosaccharide to about 7,000,000 kD.

10. The method of claim 9, wherein the physiologically acceptable salt of hyaluronic acid is a sodium salt.

11. The method of claim 10, wherein the molecular weight of the sodium salt of hyaluronic acid is between about 600 kD and about 1700 kD.

12. The method of claim 11, wherein the concentration of the sodium salt of hyaluronic acid is between about 10 ppm and about 1000 ppm in the blood.

13. A method to increase aortic blood flow, increase arterial blood flow, increase venous blood flow, decrease blood pressure, decrease peripheral vascular resistance, diminish the development of atherosclerosis, and/or prevent lethality of hemorrhagic shock, comprising the step of administering a therapeutically acceptable pharmaceutical composition comprising hyaluronic acid or a physiologically acceptable salt thereof and a pharmaceutically acceptable carrier to a subject in need thereof.

14. The method of claim 13, wherein the physiologically acceptable salt of hyaluronic acid is a sodium salt.

15. The method of claim 13, wherein the molecular weight of the hyaluronic acid or physiologically acceptable salt thereof is from that of an oligosaccharide to about 7,000,000 kD.

16. The method of claim 15, wherein the physiologically acceptable salt of hyaluronic acid is a sodium salt.

17. The method of claim 17, wherein the molecular weight of the sodium salt of hyaluronic acid is between about 600 kD and about 1700 kD.

18. The method of claim 11, wherein the concentration of the sodium salt of hyaluronic acid is between about 10 ppm and about 1000 ppm in the blood.

19. A packaged pharmaceutical comprising
hyaluronic acid or a physiologically acceptable salt thereof; and
instructions to use said hyaluronic acid or a physiologically acceptable salt thereof to increase aortic blood flow, increase arterial blood flow, increase venous blood flow, decrease blood pressure, decrease peripheral vascular resistance, diminish the development of atherosclerosis, and/or prevent lethality of hemorrhagic shock.

20. The packaged pharmaceutical of claim 19, wherein the physiologically acceptable salt of hyaluronic acid is a sodium salt.

21. The packaged pharmaceutical of claim 19, wherein the molecular weight of the hyaluronic acid or physiologically acceptable salt is from that of an oligosaccharide to about 7,000,000 kD.

22. The packaged pharmaceutical of claim 21, wherein the physiologically acceptable salt of hyaluronic acid is a sodium salt.

23. The packaged pharmaceutical of claim 22, wherein the molecular weight of the sodium salt of hyaluronic acid is between about 600 kD and about 1700 kD.

24. The packaged pharmaceutical of claim 23, wherein the concentration of the sodium salt of hyaluronic acid is between about 10 ppm and about 1000 ppm in the blood.

25. A packaged pharmaceutical comprising a pharmaceutical composition comprising:
hyaluronic acid or a physiologically acceptable salt thereof and a pharmaceutically acceptable carrier; and
instructions to use said hyaluronic acid or a physiologically acceptable salt thereof to increase aortic blood flow, increase arterial blood flow, increase venous blood flow, decrease blood pressure, decrease peripheral vascular resistance, diminish the development of atherosclerosis, and/or prevent lethality of hemorrhagic shock.

26. The packaged pharmaceutical of claim 25, wherein the physiologically acceptable salt of hyaluronic acid is a sodium salt.

27. The packaged pharmaceutical of claim 25, wherein the molecular weight of the hyaluronic acid or physiologically acceptable salt thereof is from that of an oligosaccharide to about 7,000,000 kD.

28. The packaged pharmaceutical of claim 27, wherein the physiologically acceptable salt of hyaluronic acid is a sodium salt.

29. The packaged pharmaceutical of claim 28, wherein the molecular weight of the sodium salt of hyaluronic acid is between about 600 kD and about 1700 kD.

30. The packaged pharmaceutical of claim 29, wherein the concentration of the sodium salt of hyaluronic acid is between about 10 ppm and about 1000 ppm in the blood.
